(19) United States
(12) Patent Application Publication
(10) Pub. No.: US 2024/0335115 A1
(43) Pub. Date: Oct. 10, 2024

(54) METHOD AND DEVICE FOR TESTING DECISION UNCERTAINTY

(71) Applicant: Beijing Normal University, Beijing (CN)

(72) Inventor: Xiaohong WAN, Beijing (CN)

(21) Appl. No.: 18/576,012

(22) PCT Filed: Apr. 19, 2022
(86) PCT No.: PCT/CN2022/087541

§ 371 Date: Jan. 2, 2024

(30) Foreign Application Priority Data
Jul. 2, 2021 (CN) 202110753633.X

(51) Int. Cl. A61B 5/00; A61B 5/055

(52) U.S. Cl. CPC A61B 5/0042; A61B 5/055

(57) ABSTRACT

Embodiments of the present disclosure provide a testing method and device on decision uncertainty, the method including: acquiring brain imaging data of a subject in a period from its receiving a target problem to its making a judgment on the target problem; extracting brain activity signals from a region of interest (ROI) in the brain imaging data, the ROI including at least one region among the anterior cingulate cortex, the lateral frontopolar cortex, and the ventral striatum; and obtaining, based on the brain activity signals, a first test result reflecting a degree of certainty of the subject on correctness of the judgment.

The present disclosure claims priority to Chinese Patent Application No. 202110753633.X entitled “Testing Method, Apparatus and Device on Decision Uncertainty” filed with the CNIPA on Jul. 2, 2021, which is hereby incorporated by reference in its entirety.

### TECHNICAL FIELD

Embodiments of the present disclosure relate to the technical field of testing on decision uncertainty, and specifically to a testing method and device on decision uncertainty.

### BACKGROUND

Metacognition, i.e., the cognition of cognition, is an advanced status of cognition. In a decision-making process that requires consumption of cognitive resources, decision uncertainty, as a kind of metacognition, spontaneously emerges after generation of decision-making results, reflects the degree of certainty on the correctness of the decision-making results. Decision uncertainty, as a part of metacognition, is a certain status inside the brain, and is difficult to be measured with objective indicators. Therefore, how to acquire decision uncertainty has become an urgent technical challenge for those skilled in the art to solve.

### SUMMARY

One aim of embodiments of the present disclosure is to provide a new technical solution for testing on decision uncertainty.

According to a first aspect of an embodiment of the present disclosure, an embodiment of a testing method on decision uncertainty is provided, including:
Acquiring brain imaging data of a subject in a period from its receiving a target problem to its making a judgment on the target problem;
Extracting brain activity signals from a region of interest (ROI) in the brain imaging data, the ROI including at least one region among the anterior cingulate cortex, the lateral frontopolar cortex, and the ventral striatum; and
Obtaining, based on the brain activity signals, a first test result reflecting a degree of certainty of the subject on correctness of the judgment.

Optionally, said “obtaining, based on the brain activity signals, a first test result reflecting a degree of certainty of the subject on correctness of the judgment” includes:
Inputting the brain activity signals into a pre-constructed test model to obtain a first test result reflecting a degree of certainty of the subject on correctness of the judgment, wherein the test model reflects a mapping relationship between the brain activity signals of the ROI and the first test result.

Optionally, a step of constructing the test model includes:
Constructing a test experiment based on a preset test problem and an experimental paradigm to test the subject;
Acquiring a first trial set and a second trial set from the test experiment, the first trial set being a set of experimental trials with complete certainty of the subject on correctness of its judgment, the second trial set being a set of experimental trials with complete uncertainty of the subject on a result of its judgment;
Constructing a first training sample set with certainty on judgment correctness based on brain activity signals extracted from the first trial set;
Constructing a second training sample set with uncertainty on judgment correctness based on brain activity signals extracted from the second trial set; and
Training model parameters of a basic model corresponding to the test model based on the first training sample set and the second training sample set, to obtain the test model.

Optionally, the experimental paradigm is used for characterizing a testing step for each experimental trial in the testing experiment, the testing step including:
Outputting the test problem, and continuously collecting brain imaging data of the subject through a brain imaging collection device, the test problem including a first test problem and a second test problem, wherein the first test problem is less than the second test problem in a difficulty rating in determining a judgment result; and
Receiving a second judgment result made by the subject in its judgment regarding the test problem, and determining whether the second judgment result is correct based on an acquired answer to the test problem.

Optionally, the first test problem is a first random dot animation, and the second test problem is a second random dot animation; wherein a difficulty rating is embodied as a coherence value of a random dot animation, which coherence value is a ratio of a quantity of points in the random dot animation moving in a preset direction to a total quantity of moving points in the random dot animation; the smaller the coherence value, the greater the difficulty rating; and wherein

A coherence value of the second random dot animation is 0, and a coherence value of the first random dot animation is a preset value greater than 0.

Optionally, a step of determining the preset value includes:
Constructing a random dot animation test set, which includes random dot animations with different coherence values;
Testing the subject using the experimental paradigm and based on the random dot animation test set;
Obtaining correct rates of the judgment of the subject corresponding to the random dot animations with different coherence values; and
Taking a coherence value of the random dot animations corresponding to a correct rate that meets preset conditions as the preset value.

Optionally, acquiring a first trial set and a second trial set from the test experiment includes:
Taking experimental trials corresponding to a first test problem and correctly judged by the subject as the first trial set; and
Taking experimental trials corresponding to a second test problem as the second trial set.

Optionally, before acquiring brain imaging data of a subject in a period from its receiving a target problem to its making a judgment on the target problem, the method further includes:
Outputting a target problem, and continuously collecting brain imaging data of the subject through a brain imaging collection device;

Said “acquiring brain imaging data of a subject in a period from its receiving a target problem to its making a judgment on the target problem” includes:
In the case that a first judgment result made by the subject in its judgment regarding the target problem is received, acquiring, from collected bring imaging data, brain imaging data in a period from outputting the target problem to receiving the first judgment result.

Optionally, after obtaining a first test result reflecting a degree of certainty of the subject on correctness of the judgment, the method further includes:
Acquiring self-confidence level information input by the subject regarding the judgment, the self-confidence level information reflecting a degree of certainty as reported by the subject on correctness of the judgment;
Comparing a degree of certainty reflected by the first test result with a degree of certainty reflected by the self-confidence level information, to obtain a comparison result; and
Obtaining a second test result based on the comparison result; wherein in the case that the degree of certainty reflected by the first test result is inconsistent with the degree of certainty reflected by the self-confidence level information, the second test result indicates that the subject is lying with an untruthful expression of intent.

According to a second aspect of an embodiment of the present disclosure, an embodiment of a testing device on decision uncertainty is provided, including: a processor; and a memory configured for storing a computer program, the computer program being configured to control the processor to execute a testing method of any instance of the first aspect of an embodiment of the present disclosure.

According to a third aspect of an embodiment of the present disclosure, an embodiment of a computer readable storage medium is provided having a computer program stored thereon, which computer program implements a testing method on decision uncertainty of any instance of the first aspect of an embodiment of the present disclosure when executed by a processor.

One advantage of the embodiments of the present disclosure is that the test method provided in the present disclosure may obtain a degree of certainty of the subject on correctness of its judgment, that is, the uncertainty in decision-making of the subject.

Another advantage of the embodiment of the present disclosure is: the first test result reflects a degree of certainty of the subject on correctness of its judgment, and the self-confidence level information reflects a degree of certainty as reported by the subject on correctness of its judgment: in the case that a degree of certainty reflected by a self-confidence level information is inconsistent with a degree of certainty reflected by a first test result, it is indicated that the subject is lying with an untruthful expression of intent regarding degree of certainty on correctness of its judgment. Therefore, the testing method of the present disclosure may also be used for inspecting whether the subject has made an untruthful expression of intent.

Other features and advantages of the present disclosure will become apparent and readily understood from the description of the embodiments thereof in combination with the drawings hereinafter.

### BRIEF DESCRIPTION OF THE DRAWINGS

The drawings, which are incorporated in and constitute a part of the specification, illustrate embodiments of the specification and, together with the description, serve to explain the principles of the specification.

FIG. 1 is a schematic structural illustration of a system for implementing the testing method on decision uncertainty of an embodiment of the present disclosure;

FIG. 2 is a flow diagram of the testing method on decision uncertainty according to an embodiment;

FIG. 3 is a flow diagram of an example of the testing method on decision uncertainty according to an embodiment;

FIG. 4 is a functional block diagram of an apparatus according to an embodiment;

FIG. 5 is a schematic illustration of a hardware structure of the testing device on decision uncertainty according to an embodiment.

### DETAILED DESCRIPTION

Various exemplary embodiments of the present disclosure will now be described in detail with reference to the accompanying drawings. It should be noted that unless specifically stated otherwise, the relative arrangement, numerical expressions and values of the components and steps set forth in the embodiments do not limit the scope of the present disclosure.

The following description of at least one exemplary embodiment is actually only illustrative, and in no way intended to limit the present disclosure and its application or use.

The techniques, methods, and equipment known to persons of ordinary skill in the relevant fields may not be discussed in detail, however, the techniques, methods, and equipment should be regarded as part of the specification where appropriate.

In all the examples shown and discussed herein, any specific value should be interpreted as merely exemplary rather than limiting. Therefore, other examples of the exemplary embodiment may have different values.

It should be noted that similar reference numerals and letters indicate similar items in the following drawings. Therefore, once an item is defined in one figure, it does not need to be further discussed in subsequent figures.

### <Hardware Configuration>

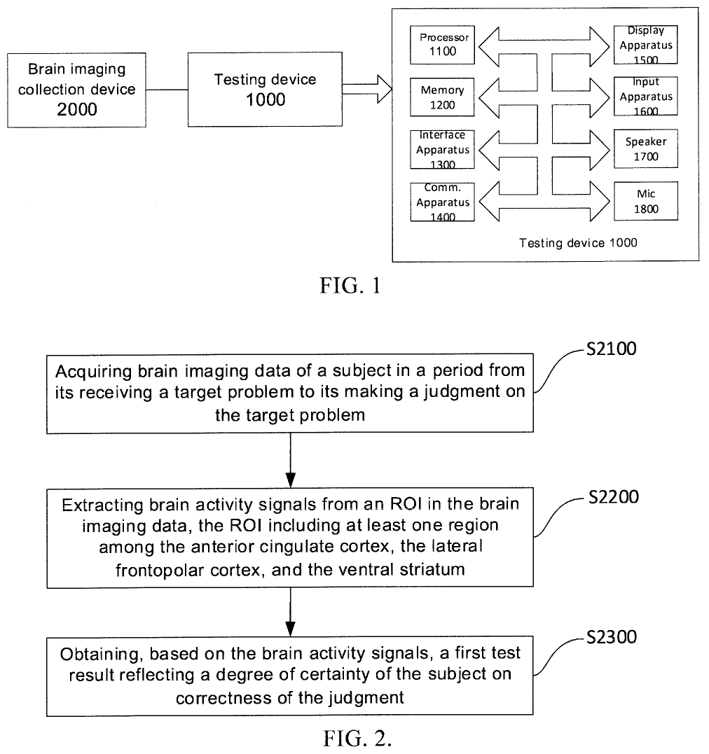

FIG. 1 is a schematic structural illustration of a system for implementing the testing method on decision uncertainty of an embodiment of the present disclosure.

As shown in FIG. 1, the system includes a testing device 1000 and a brain imaging collection device 2000. The testing device 1000 and the brain imaging collection device 2000 may be connected by wire or wirelessly.

The brain imaging collection device 2000 may be a magnetic resonance device, an electroencephalograph device, a magnetoencephalogram device, or any other device capable of implementing collection of brain imaging data, which is not limited here.

The testing device 1000 may be any electronic device with computing capability, such as a smartphone, a laptop, a desktop computer, a tablet computer, or a server, which is not limited here.

The testing device 1000 may include, but is not limited to, a processor 1100, a memory 1200, an interface apparatus 1300, a communication apparatus 1400, a display apparatus 1500, an input apparatus 1600, a speaker 1700, a microphone 1800, etc. The processor 1100 may be a central processing unit (CPU), a graphics processing unit (GPU), a microcontroller unit (MCU), etc., and is used for executing computer programs, which may be written using instruction sets such as x86, Arm, RISC, MIPS, SSE, etc. The memory 1200 may include, for example, a read-only memory (ROM), a random access memory (RAM), and a non-volatile memory such as a hard disk. The interface apparatus 1300 may include, for example, a USB interface, a serial interface, and a parallel interface. The communication apparatus 1400 may be capable of wired communication using optical fibers or cables, or wireless communication, and may specifically include WIFI network communication, Bluetooth communication, 2G/3G/4G/5G communication, etc. The display apparatus 1500 is, for example, a liquid crystal display screen, a touch display screen, etc. The input apparatus 1600 may include, for example, a touch screen, a keyboard, a body sensing input, etc. The speaker 1700 is used for outputting audio signals. The microphone 1800 is used for collecting audio signals.

As applied to an embodiment of the present disclosure, the memory 1200 of the testing device 1000 is used for storing a computer program, the computer program is used for controlling the processor 1100 to operate to implement the method according to an embodiment of the present disclosure. One skilled in the art may design the computer program according to technical solutions as disclosed in the present disclosure. How the computer program controls the processor to operate is well known in the art and will not be described in detail here. The test device 1000 may be installed with intelligent operation systems (such as Windows, Linux, Android, IOS, etc.) and application software.

Those skilled in the art should understand that although a plurality of apparatuses of the test device 1000 are shown in FIG. 1, the test device 1000 of the embodiment of the present disclosure may involve only some of the apparatuses, for example, only involving the processor 1100 and the memory 1200.

### Method Embodiment

FIG. 2 is a flow diagram of the testing method on decision uncertainty according to an embodiment, which may be implemented by a testing device.

As shown in FIG. 2, the testing method on decision uncertainty in this embodiment may include steps S2100 to S2300.

S2100: acquiring brain imaging data of a subject in a period from its receiving a target problem to its making a judgment on the target problem.

After receiving the target problem, the subject may make judgments based on the target problem through processing in the brain. During this cognitive process, the brain stores the status of cognitive activities in the process. Decision uncertainty reflects the degree of certainty that the subject has in making the correct judgment. Decision uncertainty has been shown to spontaneously emerge during the process of making judgments about a target problem. Therefore, in the above scenario, the status of cognitive activity stored in the brain necessarily contains information related to decision uncertainty.

Further, brain imaging data may be data reflecting the status of cognitive activity within the brain. Therefore, by acquiring brain imaging data of a subject in a period from its receiving a target problem to its making a judgment on the target problem; it is possible to extract decision uncertainty information stored in the brain during decision-making.

Further, the brain imaging data may be functional magnetic resonance imaging collected by a magnetic resonance device and preprocessed. It may be brain wave images collected by an electroencephalograph device and preprocessed. It may also be brain magnetic wave images collected by a magnetoencephalography device and preprocessed. In this embodiment, the brain imaging data is not specifically defined in its forms.

In step S2100, the subject receives a target problem that may cause decision uncertainty. Upon receiving a target problem that may cause decision uncertainty, some subjects may determine that their judgment is correct after making a judgment on the target problem; while others are unsure whether their judgment is correct after making a judgment on the target problem.

Taking the investigation of criminal cases as an example, the target problem that may cause decision uncertainty may be a problem preset based on the details of the case. For example, the target problem is whether the time of the crime occurred is around 5 pm, and the subject is required to make a judgment of yes or no. As persons involved in the case clearly know that the time of the crime occurred is around 5 pm, a person involved may be certain about correctness of its judgment based on its actual cognitive status. On the other hand, for an unrelated person, it does not know the specific time that the crime was occurred, and their actual cognitive status for the target problem is “unknown”, and therefore, no matter whether the unrelated person makes a judgment of “yes” or “no”, it is not certain about whether its judgment is correct.

In step S2100, the trigger time at which the subject confirms receipt of the target problem may be used as the time at which the subject receives the target problem, and the time at which the target problem is output may also be used as the time at which the subject receives the target problem, which are not limited here.

In step S2100, the time at which the first judgment result on the target problem is received from the subject may be used as the time at which the subject makes a judgment on the target problem. For example, the first judgment result input by the subject may be received through a preset sound collection device, or through a preset operation interface.

With a large number of experimental verifications, it has been proved that the subject's brain has made judgments and generated decision uncertainty within a preset time after receiving the target problem. Therefore, the preset time after outputting the target problem may also be used as the time for the subject to make a judgment on the target problem. For example, the 4^{th} second after outputting the problem may be used as the time for the subject to make a judgment on the target problem. Accordingly, in one embodiment, the time for outputting the target problem is used as the time for the subject to receive the target problem, and the 4^{th} second after outputting the problem is used as the time for the subject to make a judgment on the target problem. In this embodiment, the period from the subject's receiving a target problem to its making a judgment on the target problem, is the time period from output of the target problem to the 4^{th} second after the output of the problem.

In some embodiments, the method further includes, prior to Step S2100: outputting a target problem, and continuously collecting brain imaging data of the subject through a brain imaging collection device.

In these embodiments, the target problem may be output to the subject in response to the user-triggered start test request, and the brain imaging data of the subject may be continuously collected by the brain imaging collection device.

When outputting the target problem, it may be output in the form of video or image, and it may also be output in the form of audio. The output form of the target problem is not specifically limited in this disclosure.

In some embodiments, the step S2100 “acquiring brain imaging data of a subject in a period from its receiving a target problem to its making a judgment on the target problem” may include: in the case that the subject's judgment on the target problem has been received, acquiring brain imaging data from the collected brain imaging data in a period from the output of the target problem to the receipt of the first judgment result.

In the case that the first judgment result input by the subject is received through the preset operation interface, brain imaging data is received from the collected brain imaging data in a period from the output of the target problem to the receipt of the first judgment result input by the subject.

In the case that the preset time after the output of the target problem is taken as the time for the subject to make a judgment on the target problem, the brain imaging data in the period from the output of the target problem to the 4^{th} second after the output of the target problem is acquired at or after the 4^{th} second after the output of the target problem.

Step S2200: extracting brain activity signals from an ROI (region of interest) in the brain imaging data, the ROI including at least one region among the anterior cingulate cortex, the lateral frontopolar cortex, and the ventral striatum.

Research has found that the brain regions associated with storage of uncertainty information in decision-making include the anterior cingulate cortex, the lateral frontopolar cortex, and the ventral striatum. As such, at least one region among the anterior cingulate cortex, lateral frontopolar cortex, and ventral striatum is selected as the ROI.

In some embodiments, in order to improve accuracy of test results on uncertainty in decision-making, the ROI is matched to the size thereof. Specifically, the anterior cingulate cortex includes 341 voxels centered at the MNI152 coordinates (−3, 22, 38). The lateral frontopolar cortex includes 342 voxels centered at the MNI152 coordinates (−30, 56, 8). The ventral striatum includes 343 voxels centered at the MNI152 coordinates (+10, 10, −6). Here, the MNI152 coordinates are the coordinates in the standard space MNI152NLin6Asym.

Further, brain activity signals from the ROI are extracted from brain imaging data, and serve as the data for testing on decision uncertainty.

Step S2300: obtaining, based on the brain activity signals, a first test result reflecting a degree of certainty of the subject on correctness of the judgment.

In this embodiment, the brain activity signals represent the degree of certainty of the subject on correctness of the judgment. Therefore, according to the brain activity signals, a first test result reflecting a degree of certainty of the subject on correctness of the judgment may be obtained.

The first test result reflects an actual degree of certainty that the subject has in making the correct judgment. Here, the first test result may be information that indicates certainty, or information that indicates uncertainty. For example, the number “1” may be used as information that indicates certainty, and the number “0” may be used as information that indicates uncertainty. Alternatively, the word “certain” may be used as information that indicates certainty, and the word “uncertain” may be used as information that indicates uncertainty. When the first test result is information that indicates certainty, it shows that the subject is certain that the given judgment is correct. Alternatively, when the first test result is information that indicates uncertainty, it shows that the subject is uncertain that the given judgment is correct.

In some embodiments, said “obtaining, based on the brain activity signals, a first test result reflecting a degree of certainty of the subject on correctness of the judgment” in step S2300 may include:
Inputting the brain activity signals into a pre-constructed test model to obtain a first test result reflecting a degree of certainty of the subject on correctness of the judgment; wherein the test model reflects a mapping relationship between the brain activity signals of the ROI and the first test result, and wherein the first test result reflects a degree of certainty of the subject on correctness of the judgment.

The testing model may be a classification model constructed based on any machine learning algorithm. For example, the testing model may be a support vector machine model, a regularized logistic regression model, or a decision tree model. The testing model may also be a classification model selected based on specific requirements. The testing model is not specifically limited in this application.

The test model may be a general model or a pre-trained dedicated model for the application scenario of the embodiment of the present disclosure. For the dedicated model, the method requires constructing the test model before using it. The steps of constructing the test model may include steps S2310 to S2350.

Step S2310: constructing a test experiment based on a preset test problem and an experimental paradigm to test the subject.

A test problem is any problem that may cause decision uncertainty. For example, a test problem may be a perception-based decision-making task, such as a random dot animation, where the subject is required to judge the direction of motion of the majority of points (net motion direction) in a random dot animation (RDK) shown on the screen. A test problem may be a rule-based decision-making task, such as Sudoku, where positions of a few numbers have been given in advance, while numbers in the other blank squares require the subject to fill in through logical reasoning. According to the rule of Sudoku, the numbers in each square blank are unique and deterministic. A test problem may also be a memory-based decision-making task. There is no specific limitation on the test problems in this application.

In step S2310, a test problem may include a first test problem and a second test problem. Here, from the perspective of whether the test subject may determine the judgment result for the test problem, for example, in determining whether the judgment result is correct or not, the first test problem is less than the second test problem in a difficulty rating in determining a judgment result. That is to say, in terms of the difficulty of determining the judgment result for the test subject, the first test problem is an easy test problem, while the second test problem is a difficult test problem. For example, the first test problem is a problem that the subject may make a correct judgment based on existing knowledge; while the second test problem is a problem that the test subject cannot make a correct judgment based on existing knowledge.

In step S2310, the experimental paradigm is used for characterizing a testing step for each experimental trial in the testing experiment.

In some embodiments, the testing steps corresponding to the experimental paradigm may include steps S2311 to S2312:
Step S2311: outputting a test problem and continuously collecting brain imaging data of the subject through a brain imaging collection device;
In step S2311, upon receiving a user-triggered test request, the test problem may be output, and brain imaging data of the test subject may be continuously collected through a brain imaging collection device. The brain imaging device may be a magnetic resonance device, a magnetoencephalography device, or an electroencephalograph device. The brain imaging device is not specifically defined in the present disclosure.

Step S2312: receiving a second judgment result made by the subject in its judgment regarding the test problem, and determining whether the second judgment result is correct based on an acquired answer to the test problem.

In step S2312, the second judgment result input by the subject may be received through a preset operation interface, and the answer to the test problem may be obtained from local storage or a server. Then the second judgment result is compared with the answer to the test problem to determine whether the second judgment result made by the subject on the test problem is correct.

Furthermore, statistical analysis may be performed on correct rates of the judgment in the test experiment, based on a record of whether the second judgment result is correct.

In some embodiments, testing steps corresponding to the experimental paradigm may also include steps S2313 to S2314:
Step S2313: Outputting reward prompt information after a preset delay after outputting the test problem.

For example, when the output test problem is the first test problem, that is, an easy test problem, the reward prompt information being output may reflect information such as correct judgments receiving rewards and incorrect judgments receiving punishments. Alternatively, when the output test problem is the second test problem, that is, a difficult problem, the reward prompt information being output may reflect information such as incorrect judgments receiving rewards and correct judgments receiving punishments.

Step S2314: after receiving the second judgment result of the subject, outputting an accumulated reward information currently obtained by the subject.

Upon completion of the test experiment, the subject may exchange the accumulated reward information obtained during the test experiment for corresponding rewards. As such, in the test experiment with output reward prompt information and cumulative reward information, the subject will make judgments based on their actual cognitive status in order to obtain more rewards, and thus accurate training samples reflecting decision uncertainty may be obtained based on the test experiment.

In some embodiments, the first test problem and the second test problem may be random dot animations, that is, the first test problem is an easy random dot animation, and the second test problem is a difficult random dot animation. When generating a random dot animation, it is possible to select a preset fraction of points and make them move at a constant speed in a preset direction, while other points are evenly distributed in various directions. The ratio of the quantity of points moving in the preset direction in the random dot animation to the total quantity of moving points therein is the coherence value, which may be preset for controlling the difficulty of the random dot animation. That is to say, in this embodiment, the difficulty rating of the test problem is embodied as the coherence value of the random dot animation: the smaller the coherence value, the greater the difficulty rating. Accordingly, a coherence value of the first test problem is greater than that of the second test problem.

In this embodiment, the coherence value of a difficult random dot animation is 0; while the coherence value of an easy random dot animation is the preset value.

When the coherence of a random dot animation is 0, all the points in the random dot animation are distributed uniformly in all directions. In this circumstance, the subject cannot determine the net direction of motion of the random dot animation. Therefore, any judgment made by the subject cannot be determined to be correct. That is, during judgment on a difficult random dot animation, the brain of the subject stores signals that reflect uncertainty about the correctness of the judgment.

The coherence value of the easy random dot animation is set to a preset value, wherein a step of acquiring the preset value may include: constructing a random dot animation test set, which comprises random dot animations with different coherence values; testing the subject using the experimental paradigm and based on the random dot animation test set; obtaining correct rates of the judgment of the subject corresponding to the random dot animations with different coherence values; and taking a coherence value of the random dot animations corresponding to a correct rate that meets preset conditions as the preset value.

In the case of testing the subject using the experimental paradigm and based on the random dot animation test set, testing of the subject may be implemented by constructing a test experiment based on the random dot animation test set and the experimental paradigm in this embodiment.

In some embodiments, a coherence value of the random dot animation corresponding to a correct rate greater than or equal to 95% may be used as the set value. In an easy random dot animation, a moving point has a definite direction of net motion, and therefore the subject may make accurate judgments. That is, during judgment on an easy random dot animation, there is a signal reflecting a correct judgment stored in the subject's brain.

Step S2320: acquiring a first trial set and a second trial set from the test experiment, the first trial set being a set of experimental trials with certainty of the subject on correctness of its judgment, the second trial set being a set of experimental trials with uncertainty of the subject on a result of its judgment.

In some embodiments, a step of acquiring a first trial set and a second trial set from the test experiment in the step S2320 may include: taking experimental trials corresponding to a first test problem and correctly judged by the subject as the first trial set; and taking experimental trials corresponding to a second test problem as the second trial set.

When the test problem is easy and the subject's judgment is correct, there will be a signal stored in the subject's brain which signal reflects determining as to a second judgment result being correct.

Nevertheless, when the test problem is difficult, there will be a signal stored in the subject's brain which signal reflects uncertainty about the second judgment result being correct.

Step S2330, constructing a first training sample set with correct judgments based on brain activity signals extracted from the first trial set.

In some embodiments, constructing the first training sample set may include steps S2331 to S2335:
Step S2331: traversing through the experimental trials.

Step S2332: from experimental trials that has been currently traversed, extracting brain imaging data of a subject in a period from its receiving the test problem to its making a judgment on the test problem.

For example, a period from the output of the test problem to the receipt of a second judgment result input by the subject through a preset operation interface may be regarded as the period from the subject's receiving of the test question to the subject's making of the judgment.

Another example is that, a period from the output of the test problem to a preset timepoint after the output of the test problem may also be regarded as the period from the subject's receiving of the test question to the subject's making of the judgment.

Step S2333: extracting brain activity signals from a region of interest (ROI) in the brain imaging data, the ROI including at least one region among the anterior cingulate cortex, the lateral frontopolar cortex, and the ventral striatum.

Step S2334: upon completion of the traversal, acquiring brain activity signals extracted from each experimental trial in the first trial set.

Step S2335: labeling brain activity signals extracted from each experimental trial in the first trial set with tags representing certainty, and obtaining a first training sample set.

Step S2340: constructing a second training sample set with uncertainty on judgment correctness based on brain activity signals extracted from the second trial set.

In step S2340, constructing the second training sample set may further include: extracting brain activity signals from each experimental trial in the second trial set; and labeling brain activity signals extracted from each experimental trial in the second trial set with tags representing uncertainty, and obtaining a second training sample set.

Reference may be made to steps S2331 to S2334 for the step of extracting brain activity signals from each experimental trial in the second trial set, which are not repeated here.

Step S2350: training model parameters of a basic model corresponding to the test model based on the first training sample set and the second training sample set, to obtain the test model.

The basic model corresponding to the test model may be a regularized logistic regression model or another classification model, which are not limited here.

In some embodiments, after Step S2300, the method may further include a step S2400 of outputting the first test result.

When the testing device is executing the method, it may output the first test result in any way, such as outputting the first test result through a display, printer, speaker, etc., or sending the first test result to other terminal devices that are in communication with the testing device, such as sending it to a smartphone that is bound to the user.

In some embodiments, after obtaining the first test result reflecting a degree of certainty of the subject on correctness of the judgment, the method may further include steps S2500-S2700:
Step S2500: acquiring self-confidence level information input by the subject regarding the judgment, the self-confidence level information reflecting a degree of certainty as reported by the subject on correctness of the judgment.

Step S2600: comparing a degree of certainty reflected by the first test result with a degree of certainty reflected by the self-confidence level information, to obtain a comparison result.

Step S2700: obtaining a second test result based on the comparison result; wherein in the case that the degree of certainty reflected by the first test result is inconsistent with the degree of certainty reflected by the self-confidence level information, the second test result indicates that the subject is lying with an untruthful expression of intent.

In some embodiments, after obtaining the second test result, the method may further include Step S2800 of outputting the second test result.

When the testing device is executing the method, it may output the second test result in any way, such as outputting the second test result through a display, printer, speaker, etc., or sending the second test result to other terminal devices that are in communication with the testing device.

### EXAMPLE

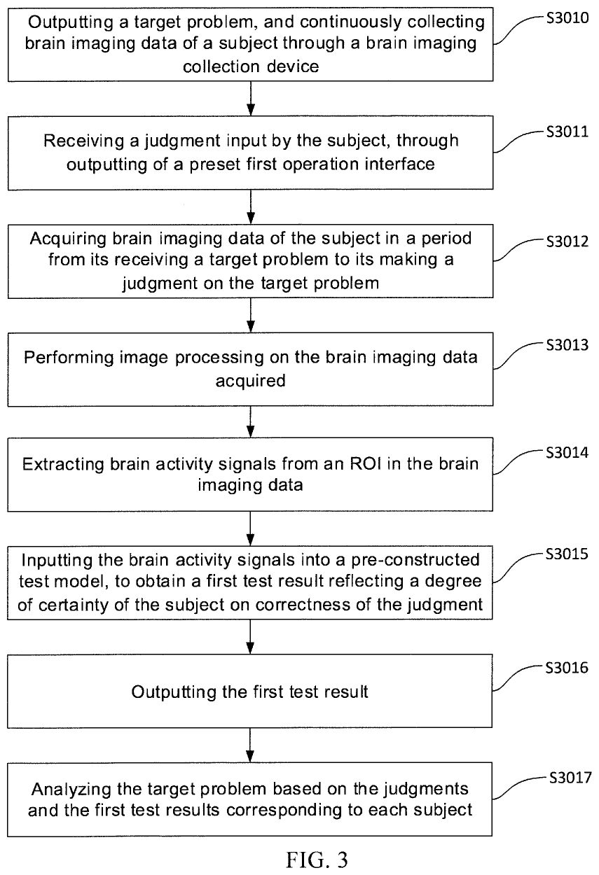

FIG. 3 is a flow diagram of an example of the testing method on decision uncertainty according to an embodiment. As shown in FIG. 3, the method may include:

Step S3010: Outputting a target problem, and continuously collecting brain imaging data of a subject through a brain imaging collection device.

In this embodiment, in response to a user-triggered request to start the test, a target problem is output to the subject through a preset interface, and brain imaging data of the subject is continuously collected through a magnetic resonance device.

In this example, the target problem that is output may be “is the company's newly proposed system reasonable?”.

Step S3011: receiving a first judgment result input by the subject, through outputting of a preset first operation interface.

In this embodiment, the preset first operation interface may be provided with a control with a name attribute of “Yes” and a control with a name attribute of “No”. Through the preset first operation interface, a judgment result of “Yes” or “No” input by the subject may be received.

Step S3012: Acquiring brain imaging data of the subject in a period from its receiving a target problem to its making a judgment on the target problem.

In this embodiment, the time at which the target problem is output is taken as the time at which the subject receives the target problem, and the time at which the first judgment result input by the subject is received is taken as the time at which the subject makes a judgment on the target problem. Functional magnetic resonance imaging in the period from the output of the target problem to the receipt of the first judgment result input by the subject is acquired.

Step S3013: Performing image processing on the brain imaging data acquired in Step S3012.

In this embodiment, the image processing step includes time calibration and head motion calibration on the acquired functional magnetic resonance imaging. The calibrated image is registered to the standard brain space. Finally, smooth filtering is performed on the registered image.

Step S3014: Extracting brain activity signals from an ROI in the brain imaging data, the ROI including at least one region among the anterior cingulate cortex, the lateral frontopolar cortex, and the ventral striatum.

In this embodiment, a BOLD signal of the ROI from the processed functional magnetic resonance imaging is extracted.

Step S3015: Inputting the brain activity signals into a pre-constructed test model, to obtain a first test result reflecting a degree of certainty of the subject on correctness of the judgment.

In this embodiment, the BOLD signal extracted in Step S3014 is input into a pre-constructed regularized logistic regression model to obtain a first test result reflecting the degree of certainty of the subject on correctness of the judgment.

Step S3016: Outputting the first test result.

In this embodiment, the first test result is output by the testing device. Specifically, the first test result is “1” or “0”. When the first test result is “1”, it indicates that the subject is certain that the judgment it gives is correct. When the first test result is “0”, it indicates that the subject is uncertain that the judgment it gives is correct.

Step S3017: Analyzing the target problem based on the judgments and the first test results corresponding to each subject.

In this example, the ratio of the number of subjects whose first test result is “1” and whose judgment result is “yes” to the total number of subjects is calculated. When the ratio is greater than a preset value, it is determined that the company's newly proposed system is reasonable. Specifically, the preset value may be 80%.

In another embodiment of the present disclosure, the method further includes steps S3018 to S3020:
Step S3018: acquiring self-confidence level information input by the subject regarding the judgment, the self-confidence level information reflecting a degree of certainty as reported by the subject on correctness of the judgment.

In this embodiment, the self-confidence level input by the subject is received by outputting a preset second operation interface. The second operation interface may be provided with a control with a name attribute of “Yes” and a control with a name attribute of “No”; wherein, the subject selecting “Yes” indicates that the subject reports its certainty on correctness of the judgment, while the subject selecting “No” indicates that the subject reports its uncertainty on correctness of the judgment.

Step S3019: comparing a degree of certainty reflected by the first test result with a degree of certainty reflected by the self-confidence level information, to obtain a comparison result.

Step S3020: obtaining a second test result based on the comparison result; wherein in the case that the degree of certainty reflected by the first test result is inconsistent with the degree of certainty reflected by the self-confidence level information, the second test result indicates that the subject is lying with an untruthful expression of intent.

In this embodiment, in the case that the first test result corresponding to the subject reflects that the subject is certain that its judgment is correct, while the self-confidence level information reflects that the subject is not certain that its judgment is correct. As such, the second test result is output as “Untruthful Expression of Intent”.

Furthermore, when analyzing the target problem, judgment data of subjects who express untruthful intent is not considered.

### Apparatus Embodiment

FIG. 4 is a functional block diagram of an apparatus according to an embodiment. As shown in FIG. 4, the testing apparatus on decision uncertainty 4000 may include a brain imaging acquisition module 4100, a data extraction module 4200, and a test result output module 4300.

The brain imaging acquisition module 4100 is configured for: acquiring brain imaging data of a subject in a period from its receiving a target problem to its making a judgment on the target problem.

The data extraction module 4200 is configured for: extracting brain activity signals from a region of interest (ROI) in the brain imaging data, the ROI including at least one region among the anterior cingulate cortex, the lateral frontopolar cortex, and the ventral striatum.

The test result output module 4300 is configured for obtaining, based on the brain activity signals, a first test result reflecting a degree of certainty of the subject on correctness of the judgment.

The above modules can also be used to perform corresponding operation steps according to corresponding embodiments provided in the above method embodiment, which will not be repeated here.

### Device Embodiment

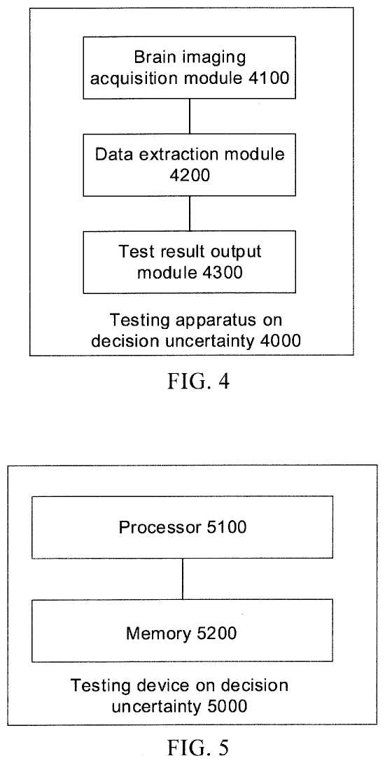

FIG. 5 is a schematic illustration of a hardware structure of the testing device on decision uncertainty according to an embodiment.

As shown in FIG. 5, the testing device on decision uncertainty 5000 includes a processor 5100 and a memory 5200. The memory 5200 is configured for storing computer programs, which computer programs are used for controlling the processor 5100 to perform a testing method on decision uncertainty in any of the above method embodiments.

The testing device on decision uncertainty 5000 may be the testing device 1000 in FIG. 1. The modules of the above testing device on decision uncertainty 5000 may be implemented by the processor 5100 in this embodiment executing computer programs stored in the memory 5200, or may be implemented by other circuit structures, which is not limited here.

One or more embodiments of the present disclosure may be a system, a method, and/or a computer program product. The computer program product may include a computer readable storage medium (or media) having computer readable program instructions thereon for causing a processor to carry out aspects of the present disclosure.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, such as the Internet, a local area network, a wide area network, and/or a wireless network. The network may include copper transmission cables, optical transmission fibers, wireless transmission, routers, firewalls, switches, gateway computers, and/or edge servers. Each computing/processing device includes a network adapter card or network interface that receives computer readable program instructions from the network and forwards the computer readable program instructions for storage in a computer readable storage medium within the respective computing/processing device.

Computer readable program instructions for carrying out operations of the present disclosure may be assembler instructions, instruction-set-architecture (ISA) instructions, machine instructions, machine dependent instructions, microcode, firmware instructions, status-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing status information of the computer readable program instructions to personalize the electronic circuitry, in order to perform aspects of the present disclosure.

Herein, aspects of the present disclosure are described with reference to flowchart illustrations and/or block diagrams of methods, apparatus (systems), and computer program products according to embodiments of the disclosure. It will be understood that the flowchart illustrations and/or block diagrams, as well as combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present disclosure. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending on the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions. It is well-known to a person skilled in the art that the implementations using hardware, using software, or using the combination of software and hardware can be equivalent with each other.

The descriptions of the various embodiments of the present disclosure have been presented for purposes of illustration, but are not intended to be exhaustive or limited to the disclosed embodiments. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the disclosed embodiments. The scope of the present disclosure is defined by the attached claims.

1. A testing method on decision uncertainty, comprising:
acquiring brain imaging data of a subject in a period from its receiving a target problem to its making a judgment on the target problem;
extracting brain activity signals from a region of interest (ROI) in the brain imaging data, the ROI including at least one of the anterior cingulate cortex, the lateral frontopolar cortex, and the ventral striatum; and
obtaining, based on the brain activity signals, a first test result reflecting a degree of certainty of the subject on correctness of the judgment.

2. The method of claim 1, wherein said obtaining,
based on the brain activity signals, a first test result reflecting a degree of certainty of the subject on correctness of the judgment comprises:
inputting the brain activity signals into a pre-constructed test model to obtain a first test result reflecting a degree of certainty of the subject on correctness of the judgment, wherein the test model reflects a mapping relationship between the brain activity signals of the ROI and the first test result.

3. The method of claim 2, wherein a step of constructing the test model comprises:
constructing a test experiment based on a preset test problem and an experimental paradigm to test the subject;
acquiring a first trial set and a second trial set from the test experiment, the first trial set being a set of experimental trials with complete certainty of the subject on correctness of its judgment, the second trial set being a set of experimental trials with complete uncertainty of the subject on a result of its judgment;
constructing a first training sample set with certainty on judgment correctness based on brain activity signals extracted from the first trial set;
constructing a second training sample set with uncertainty on judgment correctness based on brain activity signals extracted from the second trial set; and
training model parameters of a basic model corresponding to the test model based on the first training sample set and the second training sample set, to obtain the test model.

4. The method of claim 3, wherein the experimental paradigm is used for characterizing a testing step for each experimental trial in the testing experiment, the testing step comprising:
outputting the test problem, and continuously collecting brain imaging data of the subject through a brain imaging collection device, the test problem comprising a first test problem and a second test problem, wherein the first test problem is less than the second test problem in a difficulty rating in determining a judgment result; and
receiving a second judgment result made by the subject in its judgment regarding the test problem, and determining whether the second judgment result is correct based on an acquired answer to the test problem.

5. The method of claim 4, wherein the first test problem is a first random dot animation, and the second test problem is a second random dot animation; wherein a difficulty rating is embodied as a coherence value of a random dot animation, which coherence value is a ratio of a quantity of points in the random dot animation moving in a preset direction to a total quantity of moving points in the random dot animation; the smaller the coherence value, the greater the difficulty rating; and wherein
a coherence value of the second random dot animation is 0, and a coherence value of the first random dot animation is a preset value greater than 0.

6. The method of claim 5, wherein a step of determining the preset value comprises:
constructing a random dot animation test set, which comprises random dot animations with different coherence values;
testing the subject using the experimental paradigm and based on the random dot animation test set;
obtaining correct rates of the judgment of the subject corresponding to the random dot animations with different coherence values; and
taking a coherence value of the random dot animations corresponding to a correct rate that meets preset conditions as the preset value.

7. The method of claim 4, wherein acquiring a first trial set and a second trial set from the test experiment comprises:
taking experimental trials corresponding to a first test problem and correctly judged by the subject as the first trial set; and
taking experimental trials corresponding to a second test problem as the second trial set.

8. The method of claim 1, wherein, before acquiring brain imaging data of a subject in a period from its receiving a target problem to its making a judgment on the target problem, the method further comprises:
outputting a target problem, and continuously collecting brain imaging data of the subject through a brain imaging collection device;
said acquiring brain imaging data of a subject in a period from its receiving a target problem to its making a judgment on the target problem comprises:
in the case that a first judgment result made by the subject in its judgment regarding the target problem is received, acquiring, from collected bring imaging data, brain imaging data in a period from outputting the target problem to receiving the first judgment result.

9. The method of claim 1, wherein, after obtaining a first test result reflecting a degree of certainty of the subject on correctness of the judgment, the method further comprises:
acquiring self-confidence level information input by the subject regarding the judgment, the self-confidence level information reflecting a degree of certainty as reported by the subject on correctness of the judgment;
comparing a degree of certainty reflected by the first test result with a degree of certainty reflected by the self-confidence level information, to obtain a comparison result; and
obtaining a second test result based on the comparison result; wherein in the case that the degree of certainty reflected by the first test result is inconsistent with the degree of certainty reflected by the self-confidence level information, the second test result indicates that the subject is lying with an untruthful expression of intent.

10. A testing device on decision uncertainty, comprising:
a processor; and
a memory configured for storing a computer program, the computer program being configured to control the processor to execute a testing method of claim 1 on decision uncertainty.

11. A computer readable storage medium having a computer program stored thereon, which computer program implements a testing method of claim 1 on decision uncertainty when executed by a processor.
